(19) United States
(12) Patent Application Publication
(10) Pub. No.: US 2020/0367766 A1
(43) Pub. Date: Nov. 26, 2020

(54) Method and device for determining at least one physiological parameter

(71) Applicant: Gert KÜCHLER, Randersacker (DE)

(72) Inventors: Gert KÜCHLER, Randersacker (DE); Hans-Joachim MÖRSDORF, Wendelstein (DE)

(21) Appl. No.: 16/921,125

(22) Filed: Jul. 6, 2020

Related U.S. Application Data
(63) Continuation of application No. 15/534,261, filed on Jun. 8, 2017, now abandoned.

(30) Foreign Application Priority Data
Dec. 10, 2014 (DE) 10 2014 225 483.3

(51) Int. Cl. A61B 5/021; A61B 5/0225; A61B 5/00

(52) U.S. Cl. CPC A61B 5/02125; A61B 5/725; A61B 5/02255

(57) ABSTRACT

The method serves to determine at least one physiological parameter (P) of a patient. A pulse measurement signal of a pulse pressure wave propagating within the blood vessels and emanating from the heart is acquired at a pulse measurement point. A corrected pulse measurement signal is produced from the acquired pulse measurement signal by means of signal processing. The at least one physiological parameter is ascertained on the basis of the corrected pulse measurement signal. For the purposes of producing the corrected pulse measurement signal, the acquired pulse measurement signal is subjected to adaptive filtering with a dynamically adapting filter characteristic in order to compensate the influence of a reflected component of the pulse pressure wave.

### CROSS-REFERENCES TO RELATED APPLICATIONS

This application claims the priority of German Patent Application, Serial No. 10 2014 225 483.3, filed Dec. 10, 2014, pursuant to 35 U.S.C. 119(a)-(d), the content of which is incorporated herein by reference in its entirety as if fully set forth herein.

### FIELD OF THE INVENTION

The invention relates to methods and a device for determining at least one physiological parameter of a patient.

### BACKGROUND OF THE INVENTION

These days, acquiring physiological parameters is conventional and widespread in medical engineering. An example of such acquisition of a physiological parameter lies in the continuous measurement of the arterial blood pressure. Such a device and associated acquisition method which make do without the known inflatable arm cuff with the pressure sensor operating according to the Riva-Rocci principle are described in DE 10 2005 014 048 B4. The acquisition method is based on the evaluation of the pulse transit time (PTT). In so doing, the transit time of the pulse pressure wave from the heart to the periphery, for example to one of the fingers, is determined for each heartbeat. The R-wave of the ECG serves as start time of the transit time measurement and the time at which the pulse measurement signal—in particular acquired by photoplethysmography or pulse oximetry—has the maximum gradient at the periphery, i.e., for example, at the finger, serves as end time. The pulse wave speed (PWG) and, therefrom, the blood pressure, which ultimately is of interest, are ascertained from the transit time thus acquired, taking into account further parameters such as e.g. the body height. This blood pressure measurement device has proven its worth in practice. It operates very well in most cases of application. However, measurement errors occur from time to time, and so there is need for improvement.

Furthermore, DE 10 2007 024 072 A1 describes a method for displaying and evaluating ECG signals and respiration-dependent signals. Physiological parameters such as the blood pressure and oxygen saturation are acquired, the latter using a pulse oximeter. A 50 Hz filter is used to remove bothersome artifacts, such as e.g. a possible 50 Hz system hum. Moreover, use is made of a band-pass filter which masks irrelevant frequency components of the acquired pulse measurement signal.

DE 689 25 988 T2 describes a method for compensating distortions in a pulse oximeter. The distortions may be caused by artifacts as a consequence of local changes in the blood volume, by transient saturation or by blood volume artifacts. The compensation is at least partly carried out by means of frequency filtering.

DE 601 30 395 T2 describes an apparatus for monitoring the progression of the pathological state of a patient with heart failure. The apparatus comprises means for acquiring a physiological signal which is an indicator for an amplitude of an arterial pulse of the patient. In so doing, a pulse measurement signal is, inter alia, also acquired by pulse oximetry and said pulse measurement signal is subjected to broadband and narrowband filtering.

DE 60 2004 000 513 T2 describes a system for spectroscopic analysis of blood components. The analysis system comprises a signal processor which extracts a photoplethysmographic signal corresponding to a specific wavelength from an electrical signal. The system comprises an amplifier and filter unit which eliminates a noise component from an amplified electrical signal.

DE 10 2006 022 120 A1 describes a signal processing method, which finds use in plethysmogram-based measurement methods for the purposes of a low susceptibility to errors in the case of ambient light interferences and electromagnetic interferences. The signal processing method also comprises various frequency filters, but these are closely linked to the employed specific spread spectrum modulation/demodulation.

DE 198 29 544 C1 describes an apparatus for non-invasive blood pressure measurement. A variable linked to the blood flow or the blood flow speed, for example, is measured by means of ultrasound or laser Doppler technology. The signal processing disposed downstream of the measurement value acquisition also comprises filtering for removing artifacts and other interferences.

EP 2 491 856 A1 describes a method and a device for pulse detection, wherein a noise component in the acquired pulse measurement signal, which can be traced back to a body movement, is eliminated by means of adaptive filtering. To this end, provision is also made, inter alia, of a separate sensor which captures the body movement.

US 2014/0 288 445 A1 describes a method and a device for acquiring blood pressure. A reflected wave is acquired and used to validate an acquired pulse signal.

DE 698 35 843 T2 describes a further method and a further device for examining pulse waves.

### SUMMARY OF THE INVENTION

Now, an object of the invention consists of specifying a method of the type set forth at the outset with an acquisition quality that is improved in relation to the prior art.

The method according to the invention is one in which a pulse measurement signal of a pulse pressure wave propagating within the blood vessels and emanating from the heart is acquired at a pulse measurement point, a corrected pulse measurement signal is produced from the acquired pulse measurement signal by means of signal processing, and the at least one physiological parameter is ascertained on the basis of the corrected pulse measurement signal. The acquired pulse measurement signal, for the purposes of producing the corrected pulse measurement signal, is subjected to adaptive filtering with a dynamically adapting filter characteristic in order to compensate the influence of a reflected component of the pulse pressure wave.

The pulse measurement signal of the pulse pressure wave may, in particular, be a plethysmogram.

It was recognized that adaptive filtering with a dynamically adapting filter characteristic may prevent measurement errors. Otherwise, such errors may occur, in particular, on account of the influence of the reflected (or returning) pulse pressure wave. This negative influence of the reflected pulse pressure wave may be compensated particularly efficiently if use is made not of rigid filtering but of adaptive filtering with dynamic adaptation of the filter characteristic. Here, the filter characteristic of the adaptive filtering may be adapted dynamically to, in particular, physiological conditions of the patient and/or, preferably, to the current vessel state influenced, in particular, by e.g. brief activations of the autonomous nervous system as well.

A configuration in which the acquired pulse measurement signal is decomposed into measurement sections which can respectively be assigned to a heartbeat, a corrected section is ascertained from each measurement section by means of adaptive filtering, and the corrected sections thus produced are composed to form the corrected pulse measurement signal is expedient. As a result thereof, a very accurate correction of the acquired pulse measurement signal is possible. Then, in particular, the correction is adapted to the conditions prevalent in the respective measurement section. It was recognized that, by all means, these conditions may change from measurement section to measurement section, and so a signal correction carried out measurement section by measurement section is advantageous.

In accordance with a further expedient configuration, the relevant measurement section of the acquired pulse measurement signal (PM) is converted into an initial frequency signal by means of a transformation into the frequency domain. Furthermore, the initial frequency signal is used for adapting the filter characteristic and then subjected to adaptive filtering with the adapted filter characteristic, wherein a corrected frequency signal is formed, said corrected frequency signal being converted into the corrected section by means of a back transformation into the time domain. Interfering signal components caused by the reflected pulse pressure wave may be extracted and masked very efficiently in the frequency domain, with the frequency filtering advantageously being adapted to the respectively prevalent conditions which, in particular, were identified on the basis of the captured pulse measurement signal or the initial frequency signal obtained therefrom.

In accordance with a further expedient configuration, the adaptive filtering is carried out as adaptive low-pass filtering with a variable low-pass cutoff frequency. This type of filtering was found to be very efficient.

In accordance with a further expedient configuration, the adaptive filtering is carried out as adaptive low-pass filtering with a variable low-pass cutoff frequency and the amplitude maxima of the initial frequency signal are determined. Furthermore, the current value of the low-pass cutoff frequency is ascertained on the basis of the quotient of the second amplitude maximum to the third amplitude maximum, i.e., in particular, on the basis of the quotient

$$\frac{{2}^{nd} maximum}{{3}^{rd} maximum} ,$$

in order to adapt the filter characteristic. It was recognized that the second and third amplitude maxima of the initial frequency signal may be used particularly well as a measure for the currently prevalent conditions, in particular in respect of the influence of the reflected pulse pressure wave. Therefore, these two maxima may also be used very well and advantageously for adapting the filter characteristic, in particular the current low-pass cutoff frequency.

In accordance with a further expedient configuration, a frequency value of the second amplitude maximum is used as current value of the low-pass cutoff frequency if the quotient of the second amplitude maximum to the third amplitude maximum at most equals a quotient threshold, i.e., if, in particular,

$$\frac{{2}^{nd} maximum}{{3}^{rd} maximum} \leq quotient threshold$$

applies, and otherwise, i.e., if, in particular,

$$\frac{{2}^{nd} maximum}{{3}^{rd} maximum} > quotient threshold$$

applies, a frequency value of the third amplitude maximum is used as current value of the low-pass cutoff frequency, wherein the quotient threshold lies in the range between 2.0 and 3.5, in particular between 2.5 and 3.0, preferably at approximately 2.8. It was recognized that the currently prevalent conditions are reproduced and taken into account very well if the selection of the low-pass cutoff frequency is made dependent on the relationship between the quotient of the second and third amplitude maxima and the aforementioned quotient threshold. Depending on the result of this quotient check, the frequency of the second amplitude maximum or of the third amplitude maximum, i.e. the frequency at which the second amplitude maximum or the third amplitude maximum lies, is selected as low-pass cutoff frequency.

In accordance with a further expedient configuration, the method is used during a calibration. Measurement errors occurring during the calibration are particularly serious as they also have a negative influence on the measurement results subsequently acquired during normal measurement operation. Therefore, it is expedient to obtain a particularly high measurement accuracy during the calibration and, in particular, exclude or at least reduce as much as possible the adverse influences of the reflected pulse pressure wave.

In accordance with a further expedient configuration, the reflected component of the pulse pressure wave is ascertained as a difference signal corresponding to a difference between the acquired pulse measurement signal and the corrected pulse measurement signal and, in particular, evaluated separately, preferably in order to obtain in particular additional information in respect of the speed of the pulse pressure wave (=pulse wave speed (PWG)), in respect of the pulse transit time (PTT) between the heart and the pulse measurement point or in respect of static and, in particular, dynamic properties of the cardiac system and vessel system of the patient like, for example, the state and/or the behavior of the cardiac system and vessel system. These static or dynamic properties of the cardiac system and vessel system may relate, for example, to the compliance of the vessels of the patient or the pre-ejection period (PEP). This additional information may then advantageously be used to improve the pulse wave analysis in particular, for example by compensating the pre-ejection period (PEP). Overall, the accuracy and the quality of the measurement results may be improved further on the basis of this additional information. Here, the difference signal may be ascertained either by actual formation of the difference between the acquired pulse measurement signal and the corrected pulse measurement signal in the time domain or else, for example, by back transformation into the time domain of the frequency components actually filtered out in the frequency domain, i.e. discarded or deleted frequency components, during the adaptive filtering for ascertaining the corrected pulse measurement signal. In particular, the difference signal (as a measure for the reflected component of the pulse pressure wave or component of the pulse pressure wave returning to the heart) and the corrected pulse measurement signal (as a measure for the component of the pulse pressure wave propagating away from the heart) may be used to ascertain the pulse wave speed (PWG) and/or the pulse transit time (PTT) on the basis of the pulse measurement signal only, i.e., in particular, without the aid of an ECG measurement signal. To this end, for example, a time offset is ascertained between the component of the pulse pressure wave propagating away from the heart and the component of the pulse pressure wave returning to the heart, i.e. between the corrected pulse measurement signal and the difference signal. By way of example, this may be carried out by determining a time difference between prominent and, in particular, mutually corresponding times within the corrected pulse measurement signal and the difference signal. In particular, a time at which the respective signal has a maximum gradient, preferably a maximum absolute gradient, i.e. either a maximum ascent or maximum descent, comes into question as a prominent time. Then, the pulse wave speed (PWG) and/or the pulse transit time (PTT) may be ascertained, in particular additionally taking into account the known paths, in particular from the heart to the pulse measurement point, from the heart to the reflection position and between the pulse measurement point and the reflection position. In particular, the reflected component of the pulse pressure wave has experienced at least one reflection at an extremity, for example at one of the fingertips. Especially after a renewed reflection, then in the vicinity of the heart, for example at one of the upper large arterial vessels and/or at the cardiac valve, there may be a superposition with a new component of the pulse pressure wave caused at just this instant by the heart and propagating away from the heart. If the pulse measurement point lies at one of the fingertips in particular, the decisive reflected component of the pulse pressure wave acquired there has passed over the path between the region of the heart and the extremity, the fingertip in this case, three times in particular. This path between the region of the heart and e.g. the fingertip may be determined very well and at least to a good approximation. The previously-explained advantageous ascertainment of the pulse wave speed (PWG) and/or the pulse transit time (PTT) and/or—based thereon—of the systolic or diastolic blood pressure on the basis of the pulse measurement signal only, in particular using the corrected pulse measurement signal ascertained therefrom and the difference signal likewise ascertained therefrom, but preferably without using an ECG measurement signal, also constitutes dedicated subject matter of the invention when considered on its own. This applies both to the ascertainment method per se and to a device in which this method is implemented. Such a method in accordance with this independent invention is a method for determining a pulse wave speed (PWG) and/or a pulse transit time (PTT) and/or a systolic or diastolic blood pressure, in which a pulse measurement signal of a pulse pressure wave propagating within the blood vessels and emanating from the heart is acquired at a pulse measurement point, a corrected pulse measurement signal is produced from the acquired pulse measurement signal by means of signal processing, a difference signal, symbolizing a reflected component of the pulse pressure wave and determined in particular as the difference between the pulse measurement signal and the corrected pulse measurement signal, is ascertained, and the pulse transit time between the heart and the pulse measurement point is ascertained from the corrected pulse measurement signal and the difference signal, and the pulse wave speed or the systolic or diastolic blood pressure, in particular, is also ascertained on the basis of the pulse transit time, wherein, in particular, the path of the pulse pressure wave between the heart and the pulse measurement point is taken into account.

In accordance with a further expedient configuration, the corrected pulse measurement signal is used to determine at least one of the physiological parameters of the group containing a blood pressure prevailing at the pulse measurement point, in particular situated away from the heart, a central blood pressure, a plethysmogram in the proximity of the heart, and static and, in particular, dynamic properties of the cardiac system and vessel system of the patient like, preferably, the compliance of the vessels of the patient and the pre-ejection period (PEP). Thus, using this, it is possible to acquire, advantageously even in continuous fashion, e.g. the blood pressure, in particular both at a pulse measurement point situated away from the heart, e.g. at an extremity, and in the vicinity of the heart. Using this, a dynamic reproduction of the conditions is possible. In any case, such an—in particular continuous—acquisition of the blood pressure in the vicinity of the heart has hitherto not been readily possible with other methods. It is also possible to determine a preferably continuous plethysmogram in the vicinity of the heart. Thus, overall, many physiological parameters may advantageously be ascertained on the basis of the method, some of which would otherwise not be accessible or only accessible for an acquisition with a significantly higher outlay.

A further object of the invention consists of specifying a device of the type denoted at the outset, having an acquisition quality which is improved in relation to the prior art.

The device according to the invention comprises a pulse sensor for acquiring a pulse measurement signal of a pulse pressure wave which, emanating from the heart, propagates within the blood vessels up to a pulse measurement point at which the pulse sensor is arranged, and an evaluation unit for ascertaining a corrected pulse measurement signal from the acquired pulse measurement signal by means of signal processing and for ascertaining the at least one physiological parameter on the basis of the corrected pulse measurement signal, wherein the evaluation unit is configured, for the purposes of producing the corrected pulse measurement signal, to subject the acquired pulse measurement signal to adaptive filtering with a dynamically adapting filter characteristic in order to compensate the influence of a reflected component of the pulse pressure wave.

The device according to the invention substantially has the same preferred configurations as the method according to the invention. Moreover, the device according to the invention and the preferred configurations thereof substantially offer the same advantages as already described in conjunction with the method according to the invention and the variants thereof. Here, the evaluation unit may, in particular, be part of a single structural unit or else, in particular, it may also be split among two or even more structural units.

Further features, advantages and details of the invention emerge from the subsequent description of exemplary embodiments on the basis of the drawing.

### BRIEF DESCRIPTION OF THE DRAWING

FIG. 1 shows an exemplary embodiment of a blood pressure measuring device, depicted in a block diagram, for noninvasively determining the blood pressure of a patient using a corrected pulse measurement signal,

FIG. 2 shows signal curves acquired or derived within the scope of the blood pressure measuring device in accordance with FIG. 1,

FIG. 3 shows signal curves of the pulse measurement signal in the case of different superpositions of the original pulse pressure wave and the reflected pulse pressure wave,

FIG. 4 shows a frequency spectrum of an acquired pulse measurement signal, and

FIG. 5 shows signal curves of the acquired pulse measurement signal, of the corrected pulse measurement signal and of the difference signal.

### DESCRIPTION OF THE PREFERRED EMBODIMENTS

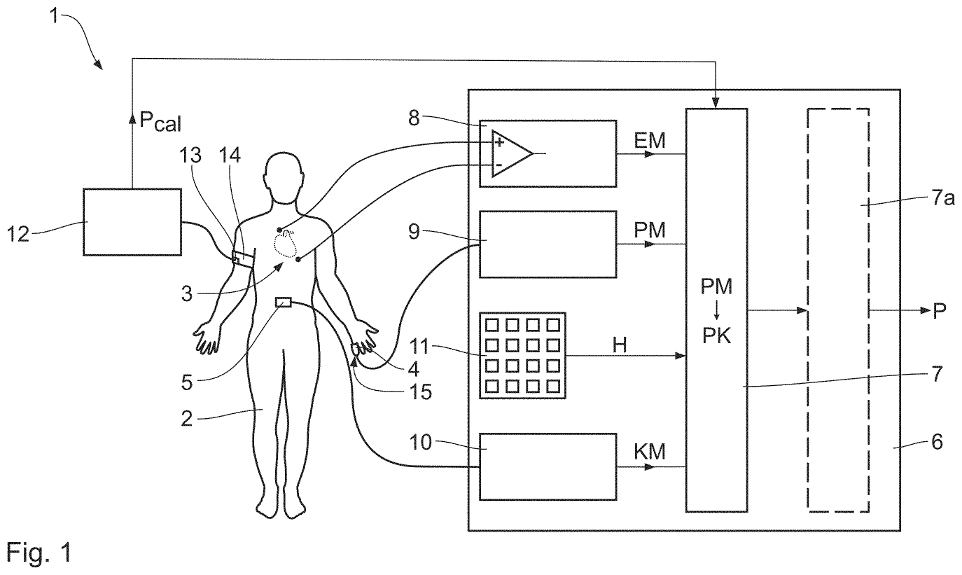

Parts corresponding to one another have been provided with the same reference signs in FIGS. 1 to 5. Details of the exemplary embodiments explained in more detail below may also constitute an invention per se or may be part of a subject of the invention.

As an example of a device for acquiring a physiological parameter, FIG. 1 shows a blood pressure measuring device 1 for noninvasive continuous determination of the (systolic or diastolic) blood pressure P of a patient 2, with the blood pressure P representing the physiological parameter to be acquired. The structure and the basic functionality of such a device based on an evaluation of the pulse transit time (PTT) are described in DE 10 2005 014 048 B4.

The blood pressure measuring device 1 contains an ECG sensor 3 comprising at least two recording electrodes, a pulse sensor 4, in particular in the form of a pulse oximeter or a photoplethysmographic sensor, and an optional body position sensor 5, in particular in the form of a 3-D acceleration sensor, which are connected to an evaluation unit 6. The evaluation unit 6 comprises a plurality of components. In addition to a first calculation unit 7 and an optional second calculation unit 7a (which is therefore only plotted using dashed lines in FIG. 1), specific subunits are present for each of the connected sensors, i.e. an ECG subunit 8, a photoplethysmographic subunit 9 and a body position subunit 10. These components of the evaluation unit 6 need not necessarily have a physically separated embodiment. They may also be realized as subprograms of software running on a signal processor or microprocessor in the evaluation unit 6. It is likewise possible for these components to be housed in a single structural unit, or else for these components to be distributed among two or even more structural units. In particular, the first calculation unit 7 and the optional second calculation unit 7a may be situated in physically separated devices. Furthermore, the evaluation unit 6 comprises input means 11, by means of which parameters, such as e.g. the body height H of the patient 2, may be entered.

A calibration unit 12 comprising a conventional blood pressure sensor 13 may be connected, at least temporarily, to the (optionally also multipart) evaluation unit 6. In the exemplary embodiment, the blood pressure sensor 13 is embodied as a Riva-Rocci blood pressure sensor comprising an inflatable arm cuff 14. A calibration blood pressure value P_{cal} ascertained during a calibration measurement by means of the blood pressure sensor 13 and the arm cuff 14 is forwarded to the evaluation unit 6.

In the exemplary embodiment in accordance with FIG. 1, the ECG sensor 3 is arranged near the heart on the rib cage of the patient 2. The pulse sensor 4 is attached to a pulse measurement point 15, to a finger of the patient 2 in the exemplary embodiment, i.e., in particular, away from the heart. A different pulse measurement point 15, such as e.g. at an ear, a toe or a limb, is likewise possible. Moreover, the pulse sensor 4 may also be embodied as a pressure sensor or as an ultrasonic sensor instead of being embodied as a photoplethysmographic sensor or as a pulse oximeter.

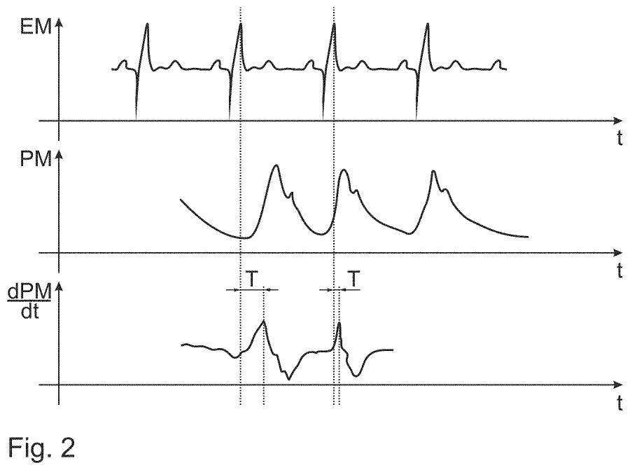

The functionality of the blood pressure measuring device 1 during normal operation emerges from the diagrams reproduced in FIG. 2, in which the signal curves are plotted over time t in each case. The ECG subunit 8 generates an electric measurement signal EM (see uppermost diagram in FIG. 2) of a cardiac current from the signals acquired by the ECG sensor 3, said electric measurement signal being fed to the calculation unit 7 for further processing purposes. The pulse sensor 4 acquires a pulse pressure wave passing the pulse measurement point 15, said pulse pressure wave propagating within the blood vessels and emanating from the heart of the patient 2. Accordingly, the photoplethysmographic subunit 9 provides a pulse measurement signal PM (see middle diagram in FIG. 2) to the calculation unit 7 on the basis of the signals acquired by the pulse sensor 4. In conjunction with the body position sensor 5, the body position subunit 10 supplies a body position signal KM (not depicted in FIG. 2) to the calculation unit 7. In particular, processing is digital in the calculation unit 7. Accordingly, the electric measurement signal EM, the pulse measurement signal PM and the body position signal KM are, in particular, digitized prior to further processing thereof.

A transit time T of the pulse pressure wave between the heart of the patient 2 and the pulse measurement point 15 is ascertained from the electric measurement signal EM and the pulse measurement signal PM in the evaluation unit 6, in particular in the first calculation unit 7 and the optional second calculation unit 7a. The time difference between the time of the so-called R-wave in the electric measurement signal EM and the time of the maximum gradient in the pulse measurement signal PM is used as transit time T. In order to ascertain the last-mentioned time more easily, the time derivative of the pulse measurement signal PM is formed (see the lowermost diagram in FIG. 2). The sought-after transit time T may then be determined by a temporal comparison of the maximum values in the measurement signal EM and in the time derivative of the pulse measurement signal PM. FIG. 2 plots the respective transit times T thus determined for two successive heartbeat cycles. The explanations above apply, in particular, to ascertaining the systolic blood pressure. When the diastolic blood pressure is ascertained, which, in principle, is carried out analogously, the time difference between the time of the minimum in the pulse measurement signal PM and the time of one of the waves in the electric measurement signal EM corresponding to the diastole are used, in particular, as transit time T.

By means of the functional relationship explained in DE 10 2005 014 048 B4, a current blood pressure P is calculated from the ascertained transit time T in the calculation unit 7, taking into account further parameters.

Thus, acquiring the time of the maximum gradient in the pulse measurement signal PM as exactly as possible is also decisive for exactly ascertaining the current value of the blood pressure P. It was found that this time cannot readily be uniquely ascertained in all constellations. This applies, in particular, if the original pulse pressure wave, i.e. the pulse pressure wave emanating from the heart, has superposed thereon a reflected or returning pulse pressure wave. Such a returning pulse pressure wave may form on account of reflections at transition regions of vessel structures and/or as a result of hydrodynamic effects. The form of the reflected pulse pressure wave may differ from that of the original pulse pressure wave depending on the vessel properties of the patient.

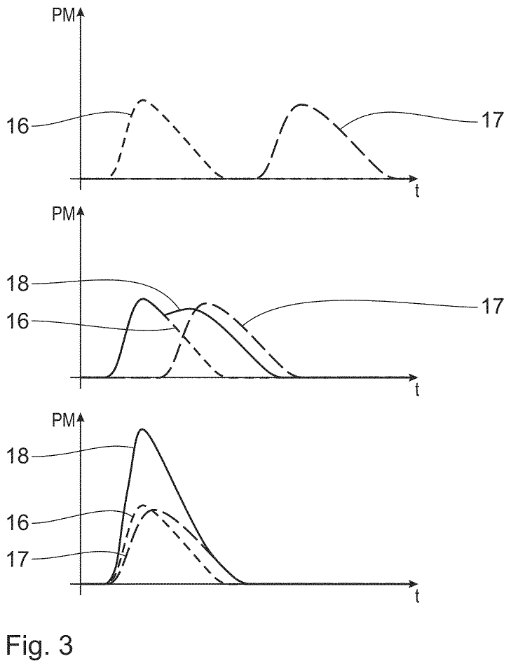

A distinction should be made between the constellations reproduced by the signal curves in accordance with FIG. 3. In the upper signal curve of FIG. 3, there is a time interval between the original pulse pressure wave 16 (depicted by a dashed line) and the reflected pulse pressure wave 17 (depicted by a dashed line), and so both pulse pressure wave components can easily be distinguished from one another and also separated from one another. In the constellation depicted in the middle diagram, the original pulse pressure wave 16 and the reflected pulse pressure wave 17 (both depicted once again by dashed lines) slightly overlap and so the pulse measurement signal PM, depicted by a full line, emerges for a pulse pressure wave 18 composed of both components. In the constellation reproduced in the lower diagram of FIG. 3, there is a far-reaching overlap between the original pulse pressure wave 16 and the reflected pulse pressure wave 17. The acquirable pulse pressure wave 18 contains the original pulse pressure wave 16 as one component and the reflected pulse pressure wave 17 as another component, with these two components no longer emerging, at least at first sight, from the acquirable pulse measurement signal PM of the (combined) pulse pressure wave 18. While the time of the maximum gradient may still be ascertained very well in the rising flank of the resultant pulse measurement signal PM in the first two constellations, this is no longer possible with the desired clarity in the third constellation reproduced in the lower diagram in accordance with FIG. 3, and so measurement errors may occur here. The effects on the measurement accuracy are particularly serious if a constellation as reproduced in the third diagram in accordance with FIG. 3 occurs during the calibration of the blood pressure measuring device 1.

In order to exclude these negative effects on the measurement accuracy, the blood pressure measuring device 1 comprises a compensation of the adverse influence of the reflected pulse pressure wave 17. In particular, the evaluation unit 6 is configured to carry out this compensation. A correction algorithm is implemented in the evaluation unit 6, said correction algorithm removing the ambiguity in relation to the time of the steepest gradient in the first rising flank of the pulse measurement signal PM. This correction algorithm is based on the discovery that higher frequency components are generated by the superposition of the reflected pulse pressure wave 17 on the original pulse pressure wave 16. Accordingly, the correction algorithm comprises adaptive frequency filtering which removes a determined higher frequency component of the pulse measurement signal PM depending on the current constellation—which, in particular, is also identified by the correction algorithm—such that said higher frequency component is not taken into account for the further signal evaluation, in particular within the scope of ascertaining the time of the maximum gradient in the first flank of the pulse measurement signal PM. A corrected pulse measurement signal PK is produced within the scope of the correction algorithm, said corrected pulse measurement signal substantially only comprising frequency components which are directly correlated with the original pulse pressure wave 16.

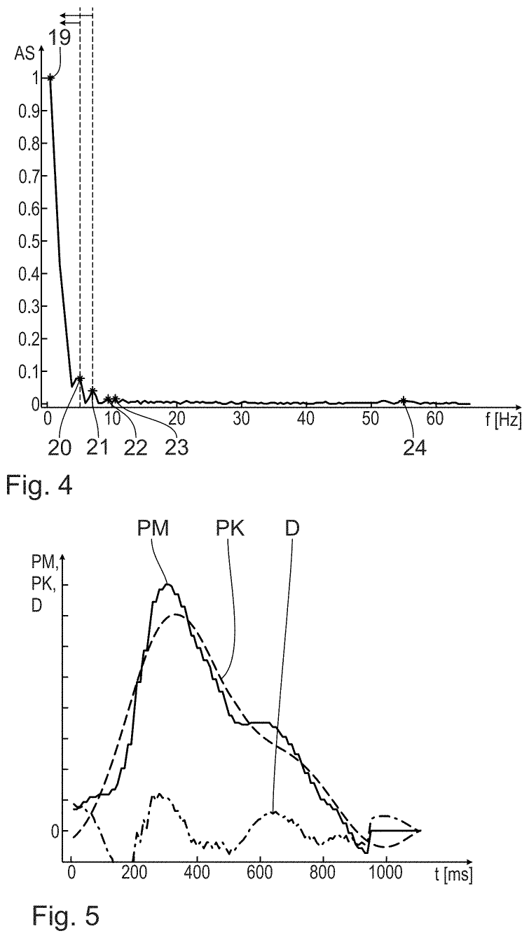

In the exemplary embodiment, this correction algorithm is realized as follows. After digitizing the recorded measurement signal, a portion which can be assigned to a heartbeat is in each case extracted from the pulse measurement signal PM as originally acquired and said portion is subjected to the actual signal correction. By way of example, the extracted portion is transformed into the frequency domain by means of a discrete Fourier transform (DFT). In order to obtain a desired frequency resolution, for example of approximately 0.5 Hz, the extracted portion of the acquired and digitized measurement signal PM is complemented with zeros at the end where necessary. The frequency signal resulting after the time-frequency transformation (=initial frequency signal) comprises a spectral amplitude component and a spectral phase component. Initially, a frequency spectrum of the amplitude component is ascertained for further evaluation purposes. An example of an amplitude spectrum AS resulting in the process is reproduced in the normalized signal curve which is plotted against the frequency f in accordance with FIG. 4. The local maxima of this amplitude spectrum AS are detected. In the image in accordance with FIG. 4, the maxima are identified by stars and denoted by the reference signs 19 to 24.

As already mentioned, the correction algorithm constitutes adaptive filtering which, in particular, has a filter characteristic which is adaptable to the current conditions. The filter characteristic is adapted on the basis of the detected maxima 19 to 24 of the amplitude spectrum AS, in particular on the basis of the second maximum 20 and third maximum 21. To this end, the amplitude value of the second maximum 20 is divided by the amplitude value of the third maximum 21. Thereafter, a check is carried out as to whether the quotient thus ascertained lies above a threshold of approximately 2.8. If this is the case, all frequency components up to and including the frequency value of the third maximum 21 are taken into account. Otherwise, i.e. if the quotient is less than or equal to the specified threshold, only frequency components up to and including the frequency value of the second maximum 20 are taken into account. The threshold may also be referred to as quotient threshold. Thus, the correction algorithm may be understood to be an adaptive low-pass filter with a variable low-pass cutoff frequency. The value of the low-pass cutoff frequency currently used for the low-pass filtering is determined by the currently prevalent conditions in this case. Even though the amplitude spectrum AS is resorted to when setting the current filter characteristic, the low-pass filtering per se acts both on the amplitude component and on the phase component of the portion of the pulse measurement signal PM transformed into the frequency domain.

Amplitude and phase components having a frequency value of at most that of the second maximum 20 of the amplitude spectrum AS are thus always taken into account. The reason lies in the discovery that, in addition to the fundamental wave, the underlying frequency components arising from damping and reflections in the vessel are also of decisive importance for the form of the original pulse pressure wave 16.

However, the current vessel state is also subject to brief activations of the autonomous nervous system. These are expressed in a vasoconstriction, leading to stiffening of the vessel walls. In order to suitably take into account these influences which at least have also been caused by the activity of the autonomous nervous system, it is expedient also to take into account higher frequency components as well, namely, in particular, up to the third maximum 21 of the amplitude spectrum AS. It was recognized that the ratio of the amplitudes of the second maximum 20 to that of the third maximum 21, as specified above, forms a good estimate for the activity of the autonomous nervous system and for other physiological conditions. Thus, in this respect, it is possible to assume, to a good approximation, that a relevant activity of the autonomous nervous system is present if the ratio lies above the aforementioned threshold. In this case, as explained above, the correction algorithm considers more frequency components within the scope of adaptive filtering.

After the low-pass cutoff frequency of the adaptive filtering has been set in accordance with the aforementioned provisions, the filtering is carried out. Here, all amplitude and phase components lying at a higher frequency than the ascertained low-pass cutoff frequency are deleted or set to zero. The residual spectrum frequency-filtered in the process (=corrected frequency signal), said residual spectrum comprising both amplitude and phase components, is thereafter transformed back into the time domain, for example by means of an inverse Fourier transform, in order thus to obtain a portion of the corrected pulse measurement signal PK. By putting together the individual corrected portions, which are thus ascertained and in each case assigned to a heartbeat, a continuous curve of the corrected pulse measurement signal PK is obtained. Since ascertaining the corrected pulse measurement signal PK is connected to a certain amount of computational outlay, use may be made of the optional second calculation unit 7a where necessary. In particular, this may be a powerful computer. However, in principle, it is also possible for all calculations for ascertaining the corrected pulse measurement signal PK to be carried out in only a single calculation unit, namely in the first calculation unit 7.

In addition to the originally acquired pulse measurement signal PM, the diagram in accordance with FIG. 5 also plots over time t the pulse measurement signal PK corrected as described above and the difference signal D formed as the difference between the originally acquired pulse measurement signal PM and the corrected pulse measurement signal PK. The original pulse measurement signal PM is reproduced using a full line, the corrected pulse measurement signal PK is reproduced using a dashed line, and the difference signal D is reproduced using a dash-dotted line. What may clearly be gathered from the signal curves reproduced in FIG. 5 is that the corrected pulse measurement signal PK has a smoothed curve in relation to the pulse measurement signal PM as originally captured. In particular, the rising flank exhibits a monotonic rising curve, and so the sought-after point with the maximum rise may also be readily ascertained and, in particular, be ascertained uniquely. Thus, the influence of the reflected pulse pressure wave 17 has been compensated for, at least to a large extent, by the described correction algorithm.

The individual portions of the pulse measurement signal PM as originally captured, which can respectively be assigned to a heartbeat, are subjected to the correction algorithm in the same manner. The corrected portions are then composed to form an overall curve of the corrected pulse measurement signal PK.

The difference signal D which, in addition to the already mentioned formation of the difference between the pulse measurement signal PM as originally acquired and the corrected pulse measurement signal PK in the time domain, may also alternatively be generated by a back transformation from the frequency domain into the time domain of the frequency components which were actually deleted or not taken into account in the aforementioned adaptive filtering is obtained as a byproduct. The difference signal D describes the returning pulse pressure wave 17. Further analyses may be carried out on the basis of the difference signal D. Thus, it is possible to obtain additional information about the state of the vessel system, for example on the basis of form and relative position of the reflected pulse pressure wave 17. Moreover, the corrected pulse measurement signal PK describes the state of the vessel system more directly than the originally captured pulse measurement signal PM, which constitutes a superposition with the component that can be traced back to the reflected pulse pressure wave 17. Consequently, the corrected pulse measurement signal PK may, additionally or alternatively, also be used for further analyses in addition to the difference signal D.

Additional information, for example in respect of the pulse wave speed and in respect of other vessel properties, such as e.g. the compliance of the vessels, may be obtained during the further analyses of the reflected pulse pressure wave 17, in particular on the basis of the difference signal D. Moreover, it is possible to estimate dynamic parameters of the cardiac system and vessel system, which dynamic parameters may then, in turn, be used for further improving the pulse wave analysis. In particular, it is then also possible to compensate the pre-ejection period (PEP), at least to a certain extent.

On account of the compensation of the reflected pulse pressure wave 17, the corrected pulse measurement signal PK reproduces the pulse pressure wave 16 originally produced by the heart much more realistically than the pulse measurement signal PM acquired far away from the heart at an extremity—at a finger in the shown exemplary embodiment. Consequently, statements in respect of the form of the pulse pressure wave 16 in the vicinity of the heart may be made on the basis of the corrected pulse measurement signal PK. This is because, at least at the beginning, there is no superposition with reflected components in the vicinity of the heart.

Moreover, the corrected pulse measurement signal PK allows an improved estimation of the central blood pressure P, i.e. of the blood pressure prevalent in the vicinity of the heart. Directly acquiring the blood pressure P in the vicinity of the heart is not possible, or at least not possible without significant outlay.

Thus, the blood pressure measuring device 1 and, in particular, the correction algorithm implemented in the evaluation unit 6 are very advantageous. The implemented adaptive filter dynamically adapts the filter characteristic thereof to the physiological conditions of the patient 2 and, in particular, to the autonomous activation thereof. The adaptive filtering thus undertaken achieves at least a significant attenuation of the adverse influences of the reflecting pulse pressure wave 17, as a result of which the blood pressure P may be ascertained more accurately. The correction algorithm may be used both during a calibration and during the actual measurement operation of the blood pressure measuring device 1.

As explained above, the compensation method however also offers numerous other options for acquiring different physiological parameters, such as e.g. the speed of the pulse pressure wave, static and/or dynamic properties of the cardiac system and vessel system, including the compliance of the vessels and the pre-ejection period. Hence, the described correction algorithm may be advantageously used not only in conjunction with a blood pressure measurement, but also when acquiring further physiological parameters. In this respect, the blood pressure measuring device 1 described above should only be understood to be exemplary. The correction algorithm may be transferred analogously to other acquisition methods and acquisition devices. The advantages described above also take effect in the same or a similar manner in these further applications.

What is claimed is:

1. A method for determining at least one physiological parameter of a patient, in which
a) a pulse measurement signal of a pulse pressure wave propagating within the blood vessels and emanating from the heart is acquired at a pulse measurement point,
b) a corrected pulse measurement signal is produced from the acquired pulse measurement signal by means of signal processing, and
c) the at least one physiological parameter is ascertained on the basis of the corrected pulse measurement signal,
wherein
d) the acquired pulse measurement signal, for the purposes of producing the corrected pulse measurement signal, is subjected to adaptive filtering with a dynamically adapting filter characteristic in order to compensate the influence of a reflected component of the pulse pressure wave.

2. A method as claimed in claim 1, wherein the acquired pulse measurement signal is decomposed into measurement sections which can respectively be assigned to a heartbeat, a corrected section is ascertained from each measurement section by means of adaptive filtering, and the corrected sections thus produced are composed to form the corrected pulse measurement signal.

3. A method as claimed in claim 2, wherein the relevant measurement section of the acquired pulse measurement signal is converted into an initial frequency signal by means of a transformation into the frequency domain, the initial frequency signal is used for adapting the filter characteristic and then subjected to adaptive filtering with the adapted filter characteristic, wherein a corrected frequency signal is formed, said corrected frequency signal being converted into the corrected section by means of a back transformation into the time domain.

4. A method as claimed in claim 1, wherein the adaptive filtering is carried out as adaptive low-pass filtering with a variable low-pass cutoff frequency.

5. A method as claimed in claim 3, wherein the adaptive filtering is carried out as adaptive low-pass filtering with a variable low-pass cutoff frequency, the amplitude maxima of the initial frequency signal are determined, and the current value of the low-pass cutoff frequency is ascertained from the quotient of the second amplitude maximum to the third amplitude maximum in order to adapt the filter characteristic.

6. A method as claimed in claim 5, wherein a frequency value of the second amplitude maximum is used as current value of the low-pass cutoff frequency if the quotient of the second amplitude maximum to the third amplitude maximum at most equals a quotient threshold, and otherwise a frequency value of the third amplitude maximum is used as current value of the low-pass cutoff frequency, wherein the quotient threshold lies in the range between 2.0 and 3.5.

7. A method as claimed in claim 5, wherein a frequency value of the second amplitude maximum is used as current value of the low-pass cutoff frequency if the quotient of the second amplitude maximum to the third amplitude maximum at most equals a quotient threshold, and otherwise a frequency value of the third amplitude maximum is used as current value of the low-pass cutoff frequency, wherein the quotient threshold lies in the range between 2.5 and 3.0.

8. A method as claimed in claim 5, wherein a frequency value of the second amplitude maximum is used as current value of the low-pass cutoff frequency if the quotient of the second amplitude maximum to the third amplitude maximum at most equals a quotient threshold, and otherwise a frequency value of the third amplitude maximum is used as current value of the low-pass cutoff frequency, wherein the quotient threshold lies at 2.8.

9. A method as claimed in claim 1, it is used during a calibration.

10. A method as claimed in claim 1, wherein the reflected component of the pulse pressure wave is ascertained as a difference signal corresponding to a difference between the acquired pulse measurement signal and the corrected pulse measurement signal.

11. A method as claimed in claim 10, wherein the reflected component of the pulse pressure wave is evaluated separately.

12. A method as claimed in claim 10, wherein the reflected component of the pulse pressure wave is evaluated separately in order to obtain information at least one of in respect of the speed of the pulse pressure wave, in respect of the pulse transit time between the heart and the pulse measurement point, in respect of static properties of the cardiac system and vessel system of the patient, in respect of dynamic properties of the cardiac system and vessel system of the patient, in respect of the compliance of the vessels of the patient and in respect of the pre-ejection period.

13. A method as claimed in claim 1, wherein the corrected pulse measurement signal is used to determine at least one of the physiological parameters of the group containing a blood pressure prevailing at the pulse measurement point, a central blood pressure, a plethysmogram in the proximity of the heart, static properties of the cardiac system and vessel system of the patient, dynamic properties of the cardiac system and vessel system of the patient and the compliance of the vessels of the patient and the pre-ejection period.

14. A device for determining at least one physiological parameter of a patient, comprising
a) a pulse sensor for acquiring a pulse measurement signal of a pulse pressure wave which, emanating from the heart, propagates within the blood vessels up to a pulse measurement point at which the pulse sensor is arranged, and
b) an evaluation unit for ascertaining a corrected pulse measurement signal from the acquired pulse measurement signal by means of signal processing and for ascertaining the at least one physiological parameter on the basis of the corrected pulse measurement signal,
wherein
c) the evaluation unit is configured, for the purposes of producing the corrected pulse measurement signal, to subject the acquired pulse measurement signal to adaptive filtering with a dynamically adapting filter characteristic in order to compensate the influence of a reflected component of the pulse pressure wave.

15. A device as claimed in claim 14, wherein the evaluation unit is configured to decompose the acquired pulse measurement signal into measurement sections which can respectively be assigned to a heartbeat, ascertain a corrected section from each measurement section by means of adaptive filtering, and compose the corrected sections thus produced to form the corrected pulse measurement signal.

16. A device as claimed in claim 15, wherein the evaluation unit is configured to convert the relevant measurement section of the acquired pulse measurement signal into an initial frequency signal by means of a transformation into the frequency domain, use the initial frequency signal for adapting the filter characteristic and then subject said initial frequency signal to adaptive filtering with the adapted filter characteristic, wherein a corrected frequency signal is formed, and the evaluation unit is further configured to convert the corrected frequency signal into the corrected section by means of a back transformation into the time domain.

17. A device as claimed in claim 14, wherein the evaluation unit is configured to carry out the adaptive filtering as adaptive low-pass filtering with a variable low-pass cutoff frequency.

18. A device as claimed in claim 16, wherein the evaluation unit is configured to carry out the adaptive filtering as adaptive low-pass filtering with a variable low-pass cutoff frequency, determine the amplitude maxima of the initial frequency signal, and ascertain the current value of the low-pass cutoff frequency from the quotient of the second amplitude maximum to the third amplitude maximum in order to adapt the filter characteristic.

19. A device as claimed in claim 18, wherein the evaluation unit is configured to use a frequency value of the second amplitude maximum as current value of the low-pass cutoff frequency if the quotient of the second amplitude maximum to the third amplitude maximum at most equals a quotient threshold, and otherwise use a frequency value of the third amplitude maximum (21) as current value of the low-pass cutoff frequency, wherein the quotient threshold lies in the range between 2.0 and 3.5.

20. A device as claimed in claim 18, wherein the evaluation unit is configured to use a frequency value of the second amplitude maximum as current value of the low-pass cutoff frequency if the quotient of the second amplitude maximum to the third amplitude maximum at most equals a quotient threshold, and otherwise use a frequency value of the third amplitude maximum as current value of the low-pass cutoff frequency, wherein the quotient threshold lies in the range between 2.5 and 3.0.

21. A device as claimed in claim 18, wherein the evaluation unit is configured to use a frequency value of the second amplitude maximum as current value of the low-pass cutoff frequency if the quotient of the second amplitude maximum to the third amplitude maximum at most equals a quotient threshold, and otherwise use a frequency value of the third amplitude maximum as current value of the low-pass cutoff frequency, wherein the quotient threshold lies at 2.8.

22. A device as claimed in claim 14, wherein the evaluation unit is configured to perform ascertaining the corrected pulse measurement signal and ascertaining the at least one physiological parameter on the basis of the corrected pulse measurement signal during a calibration of the device.

23. A device as claimed in claim 14, wherein the evaluation unit is configured to ascertain the reflected component of the pulse pressure wave as a difference signal corresponding to a difference between the acquired pulse measurement signal and the corrected pulse measurement signal.

24. A device as claimed in claim 23, wherein the evaluation unit is configured to separately evaluate the reflected component.

25. A device as claimed in claim 23, wherein the evaluation unit is configured to separately evaluate the reflected component in order to obtain information at least one of in respect of the speed of the pulse pressure wave, in respect of the pulse transit time between the heart and the pulse measurement point, in respect of static properties of the cardiac system and vessel system of the patient, in respect of the speed of the pulse pressure wave, one of in respect of the pulse transit time between the heart and the pulse measurement point and in respect of dynamic properties of the cardiac system and vessel system of the patient and in respect of the compliance of the vessels of the patient and in respect of the pre-ejection period.

26. A device as claimed in claim 14, wherein the evaluation unit is configured to use the corrected pulse measurement signal to determine at least one of the physiological parameters of the group containing a blood pressure prevailing at the pulse measurement point, a central blood pressure, a plethysmogram in the proximity of the heart, static properties of the cardiac system and vessel system of the patient, dynamic properties of the cardiac system and vessel system of the patient, the compliance of the vessels of the patient and the pre-ejection period.
